(19) United States
(12) Patent Application Publication
(10) Pub. No.: US 2015/0057720 A1
(43) Pub. Date: Feb. 26, 2015

(54) NERVE ROOT STIMULATOR AND METHOD FOR OPERATING NERVE ROOT STIMULATOR

(71) Applicant: Kwang Chon Ko, Gyeonggi-do (KR)

(72) Inventor: Kwang Chon Ko, Gyeonggi-do (KR)

(73) Assignee: Lutronic Corporation, Gyeonggi-do (KR)

(21) Appl. No.: 14/388,570

(22) PCT Filed: Mar. 27, 2012
(86) PCT No.: PCT/KR2012/002200

§ 371 Date: Sep. 26, 2014

(30) Foreign Application Priority Data
Mar. 27, 2012 (KR) 10-2012-0030850

(51) Int. Cl. A61N 1/378; A61N 1/36; A61N 1/372

(52) U.S. Cl. CPC A61N 1/3787; A61N 1/37229; A61N 1/36125
USPC 607/60; 607/61

(57) ABSTRACT

A nerve root stimulator, according to the present invention, comprises: a body which is installed at the spinal nerve root inside the human body; an antenna, which is provided on the body, for receiving a wireless frequency that is irradiated from outside of the human body; a power generation unit for generating power by using the wireless frequency that is received by the antenna; and electrodes for electrically stimulating the spinal nerve root by using the power that is generated by the power generation unit.

### BACKGROUND OF THE INVENTION

1. Field of the Invention

The present invention relates to a nerve root stimulator, and more particularly, to nerve root stimulator that is implanted in a nerve root and a dorsal root ganglion of a human body to stimulate a nerve root and a dorsal root ganglion by using a radio frequency irradiated from the outside, and a method for operating the nerve root stimulator.

2. Related Art

A method that applies an electric signal to a spinal cord for treating and managing pain of a patient is one of very old therapy methods. Although a correlation between a nervous tissue and electric energy is not completely revealed, paresthesia is induced by applying an electric pulse to the nervous tissue of a human body to reduce the pain. In addition, the electric signal is used even for treating various motor disorder symptoms. For example, the motor disorder includes tremor, dystonia, spasticity, and the like.

Since a spinal nervous tissue sends a signal for motor control or a pain signal to a muscle, when the electric signal is applied to a nerve root, the motor disorder and the pain can be treated by the paresthesia. A device that simulates the spinal cord by using the electric signal is referred to as a spinal cord stimulator.

As the known spinal cord stimulator, US Patent Publication Application No. US 20110264181, Spinal Cord Stimulator Lead Anchor is provided. The US Patent Publication Application discloses a component including a lead inserted and implanted into a spinal part of a patient, an electrode provided on the end of the lead, a pulse generator installed outside a human body and connected with the lead, and a fixing member for fixing the lead to the human body.

In the case of the spinal cord stimulator of the US Patent Publication Application configured as above, the lead needs to be inserted into the human body give electric stimulus to a spine and the electric signal needs to be transferred to a lesion through a stimulator by using the pulse generator while the lead is extended and exposed to the outside of the human body.

As a result, since the patient receives a stimulus treatment by connecting the stimulator to the lead while the lead extended from a spine part is exposed to the outside of the human body, the user feels very great inconvenience in using the stimulator.

Further, the known spinal cord stimulator performs the electric stimulus at an approximate location of a spinal cord part. However, a plurality of nerve roots positioned at the spinal cord part are connected with nerves of key parts of the human body, respectively.

Therefore, a nerve root at a specific part is stimulated to achieve a more efficient stimulus treatment, but since the known spinal cord stimulator transfers a high-energy pulse to the spinal cord part to a wide range at the approximate location, the known spinal cord stimulator is low in treatment effect, and may change the flow of a cerebral spinal fluid that flows in the spine and damage various human tissues adjacent to the nervous tissue or the nerve root due to the high-energy pulse.

### SUMMARY OF THE INVENTION

An aspect of the present invention provides a nerve root stimulator and a method for operating the nerve root stimulator having an advantage to electrically stimulate a nerve root by using a radio frequency which is irradiated from the outside to the nerve root stimulator installed in a nerve root in a human body.

An exemplary embodiment of the present invention provides a nerve root stimulator including: a body part installed in a nerve root in a human body; an antenna provided in the body part to receive a radio frequency irradiated from the outside; a power generating unit generating power by using the radio frequency received to the antenna; and an electrode stimulating the nerve root by using the power generated in the power generating unit.

The power generating unit may include a rectifying unit rectifying the radio frequency received to the antenna to a direct current, and a switching unit controlling the power provided to the electrode.

A plurality of electrodes may be provided, and the power generating unit may provide power having different sizes to the plurality of electrodes.

The nerve root stimulator may further include a demodulator demodulating a transmission signal at the radio frequency received to the antenna and a control unit controlling an operation of the power generating unit by using data transferred from the demodulator.

A plurality of antennas extended in a length direction of the body part may be included.

Another exemplary embodiment of the present invention provides a method for operating a nerve root stimulator including: receiving a radio frequency by a nerve root stimulator installed in a spinal nerve root in a human body; generating power by using the received radio frequency; and applying a current to an electrode of the nerve root stimulator contacting the nerve root by using the power.

A plurality of electrodes is provided, and power having different sizes may be provided to the plurality of electrodes.

A transmission signal may be demodulated at the received radio frequency to control the operation of the nerve root stimulator.

The nerve root stimulator may be installed at a dorsal root ganglion.

Yet another exemplary embodiment of the present invention provides a method for operating a nerve root stimulator including: receiving a radio frequency by a plurality of nerve root stimulators installed at a plurality of locations in a spinal nerve root in a human body; demodulating an identification signal from the radio frequency by the plurality of nerve root stimulators; verifying an unique identifier from the identification signal by the plurality of nerve root stimulators; generating power by using the received radio frequency by the nerve root stimulator identified by the unique identifier among the plurality of nerve root stimulators; and applying a current to an electrode of the identified nerve root stimulators by using the power.

A plurality of electrodes is provided, and power having different sizes may be provided to the plurality of electrodes.

A transmission signal may be demodulated at the received radio frequency to control the operation of the nerve root stimulator.

The nerve root stimulator may be installed at a dorsal root ganglion.

In the nerve root stimulator according to the present invention, the nerve root stimulator is fixedly installed at a spinal stimulation site in the human body, a frequency is wirelessly transmitted to the nerve root stimulator in the human body without inserting a separate lead into the human body, the nerve root stimulator receiving the radio frequency generates self-power to stimulate the corresponding stimulation site, and as a result, stimulation treatment may be performed even in a state where a patient wears the lead connecting the inside and the outside of the human body.

### BRIEF DESCRIPTION OF THE DRAWINGS

FIG. 1 is a plan view illustrating an example of a nerve root stimulator according to an exemplary embodiment of the present invention.

FIG. 2 is a cross-sectional view illustrating an example of a nerve root stimulator according to the exemplary embodiment of the present invention.

FIG. 3 is a block diagram illustrating a configuration of a nerve root stimulator according to the exemplary embodiment of the present invention.

FIG. 4 is a flowchart illustrating a method for operating a nerve root stimulator according to another exemplary embodiment of the present invention.

FIG. 5 is a diagram for describing an operation state of a nerve root stimulator according to another exemplary embodiment of the present invention.

FIG. 6 is a diagram for describing an installation state of a nerve root stimulator according to another exemplary embodiment of the present invention.

FIG. 7 is a diagram for describing an example of a using state of a nerve root stimulator according to another exemplary embodiment of the present invention.

### DESCRIPTION OF EXEMPLARY EMBODIMENTS

Hereinafter, exemplary embodiments of a nerve root stimulator and a method for operating the nerve root stimulator according to the present invention will be described with reference to the accompanying drawings. As those skilled in the art would realize, the described embodiments may be modified in various different ways, all without departing from the spirit or scope of the present invention. On the contrary, exemplary embodiments introduced herein are provided to make disclosed contents thorough and complete and sufficiently transfer the spirit of the present invention to those skilled in the art.

A nerve root stimulator according to an exemplary embodiment of the present invention is installed so that an electrode is in contact with a nerve root to be stimulated. Further, the installation of the nerve root stimulator may be performed by using a minimal invasion surgery method. In addition, a structure and a shape of the nerve root stimulator may be manufactured by various forms and shapes which are operable by the minimal invasion surgery method. Further, a shape, a position, a size, and a contact area of an electrode of the nerve root stimulator, an amplitude of a current, an operation pattern and an algorithm of the nerve root stimulator, and the like may be variously modified due to a patient's state or various other reasons.

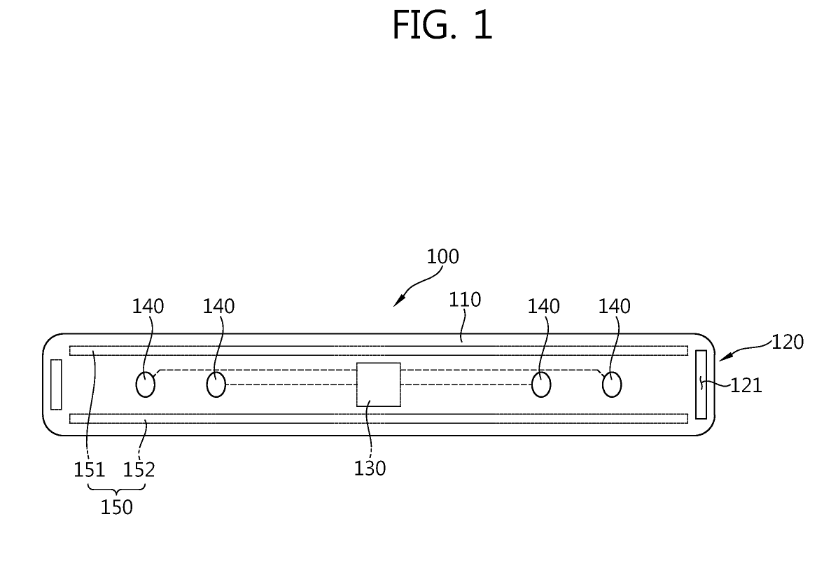

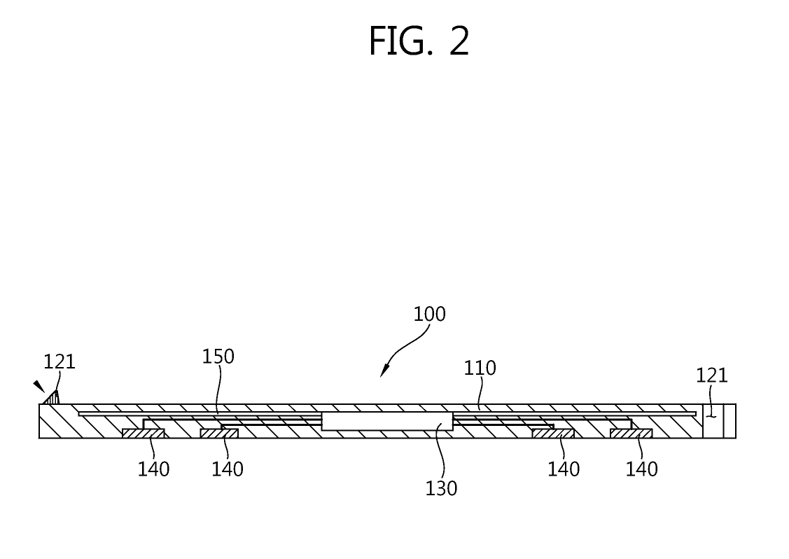

FIG. 1 is a plan view illustrating an example of a nerve root stimulator according to an exemplary embodiment of the present invention, and FIG. 2 is a cross-sectional view illustrating an example of a nerve root stimulator according to the exemplary embodiment of the present invention.

As illustrated in FIGS. 1 and 2, a nerve root stimulator 100 includes a body part 110 fixedly installed to the inside of a human body, for example, a nerve root or a dorsal root ganglion of spinal cord. The overall shape of the body part 110 may be formed in a band shape in which a width is small and a length is large. Further, a cover of the body part 110 is made of a material such as silicon which is harmless and elastic. In addition, a connection part 120 connecting both ends of the body part 110 is provided at both ends of the body part 110. The connection part 120 may be provided by a hole 121 formed at one side end of the body part 110 and an a hook 122 formed at the other side end of the body part 110 and inserted to the hole 121, and further, may be formed by various other shapes or structures. Further, the connection part 120 may be a structure such as an anchor by which the body part is connected to a tissue of the human body.

A main chip 130 is embedded in an internal center of the body part 110. A power generating unit 130 is provided in the main chip 130. In addition, at least one electrode 140 is exposed and installed on a surface contacting the bottom of the body part 110, that is, the nerve root or the dorsal root ganglion.

In addition, the electrode 140 may be mad of harmless stainless steel, titanium, and aluminum, and nano-composite thin films may be formed on a contact surface with the human body. When a plurality of electrodes 140 is installed, each electrode 140 is individually connected to the main chip 130 by wire.

The antennas 150 which are receiving units are embedded in the body part 110 at both sides of the main chip 130 inside the body part 110. The antennas 150 include a first antenna 151 extended in one length direction of the body part 110, and a second antenna 152 extended in the other length direction of the body part 110. The shapes of the antennas 151 and 152 may be formed by various shapes in order to more efficiently receive a radio frequency irradiated from the outside and transmit the radio frequency to the outside if necessary. In addition, the antenna 150 wirelessly receives a frequency transmitted in vitro and then induces a current to generate an alternating current and transfers the alternating current to the main chip 130.

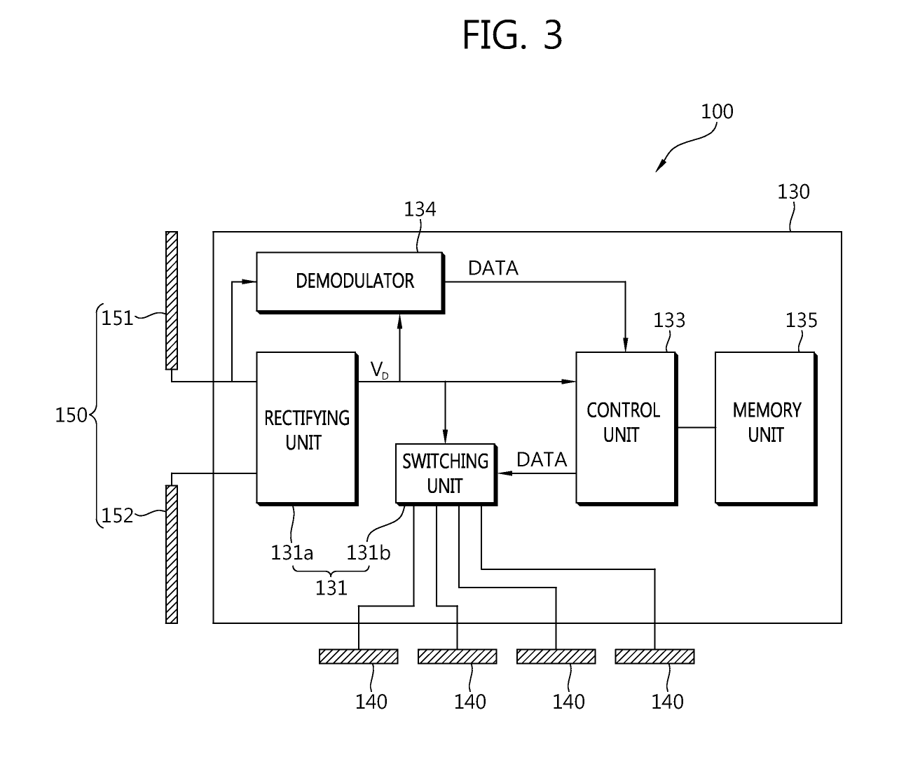

FIG. 3 is a block diagram illustrating a configuration of a nerve root stimulator according to the exemplary embodiment of the present invention. As illustrated in FIG. 3, the main chip 130 includes a power generating unit 131 wirelessly inducing the frequency received in the antenna 150 to generate power. The power generating unit 131 includes a rectifying unit 131a and a switching unit 131b which are mounted on the main chip 131.

The rectifying unit 131a rectifies the alternating current transferred from the antenna 150 to a direct current to generate the power. The rectifying unit 131a is constituted by a rectifier circuit and mounted on the main chip 130. In addition, the rectifying unit 131a and the antenna 150 may be connected to each other by an inlay method and various other methods. In addition, a smoothing circuit which is a smoothing unit may be mounted on the main chip 130 together with the rectifying unit 131a so as to maintain the current rectified in the rectifying unit 131a at a predetermined value.

Meanwhile, the current rectified in the rectifying unit 131a may be selectively or simultaneously to the switching unit 131b, a control unit 133, and a demodulator 134. That is, in order to control the supply of the current, power consumption for driving may be provided to the control unit 133 and the demodulator 134. However, all of the switching unit 131b, the control unit 133, and the demodulator 134 may be selective constituent elements. That is, in the exemplary embodiment of the present invention, the nerve root stimulator 100 may be configured to apply the current to the electrode 140 without a separate process or treatment after rectifying the frequency received wirelessly.

The switching unit 131b functions to selectively control current supply in order to supply the current to at least one electrode 140. In addition, the control unit 133 may serve to determine an operation of the switching unit 131b. In addition, the demodulator 134 serves to demodulate a signal which is modulated to a radio frequency and carried to identify a transmission signal and to transmit the demodulated signal to the control unit 133.

In addition, the control unit 133 may store various data and information included in the transmission signal, in a memory unit 135 if necessary. The transmission signal may be various. That is, the transmission signal may be a command to require an operation of the nerve root stimulator 100, a command to operate only a predetermined electrode 140, a command to operate the entire electrode 140, a command to apply the current to the electrode 140 at a predetermined voltage, and commands for an time interval or a cycle at which the current is applied to the electrode 140.

Accordingly, a plurality of nerve root stimulators 100 installed at a plurality of locations of spinal nerve roots in the human body is installed, and each of the plurality of nerve root stimulators 100 demodulates a own identification signal from the radio frequency. In addition, each nerve root stimulator 100 verifies each unique identifier from the identification signal, and thereafter, only the nerve root stimulators 100 identified by the unique identifier generate the power by using the received radio frequency to perform an electrical stimulation at a selected location. In addition, even in the case, each of the nerve root stimulators 100 includes a plurality of electrodes to apply currents having different intensities in each electrode 140.

In addition, as another exemplary embodiment, although not illustrated, the modulator is mounted on the main chip 130 together to wirelessly transmit a required signal to the outside in the main chip 130. That is, in the case where the plurality of nerve root stimulators 100 is inserted into the human body, the plurality of nerve root stimulators 100 receiving the radio frequency together may transmit to information on various parameters such as an unique identifier representing themselves, state information of the nerve root stimulator 100, and a patient's state, to the outside. In addition, in this case, as the required modulation method, a method such as backscattering modulation or impedance modulation may be adopted.

As such, the nerve root stimulator 100 according to the exemplary embodiment of the present invention is installed at the nerve root or the dorsal root ganglion to generate the power by using the wirelessly transmitted frequency and transmits the electrical signal to the nerve root or the dorsal root ganglion by the power to treat pains and movement disorders of patients. Hereinafter, a method for operating the nerve root stimulator according to the exemplary embodiment of the present invention will be described.

First, the nerve root stimulator 100 is installed at the nerve root or the dorsal root ganglion in the human body. That is, the nerve root stimulator 100 may be implanted in the nerve root or the dorsal root ganglion. In the implanting method, a back side of the human body is partially cut by minimal invasion surgery, and a catheter enters the dorsal root ganglion through the back side of the human body.

In addition, when the catheter is adjacent to the dorsal root ganglion, an operation tool in which the nerve root stimulator 100 is mounted is inserted into the end through an inner channel of the catheter. Thereafter, the nerve root stimulator 100 surrounds the dorsal root ganglion by using the operation tool and then connection parts 120 of both ends of the nerve root stimulator 100 are connected to each other, and as a result, the nerve root stimulator 100 is installed.

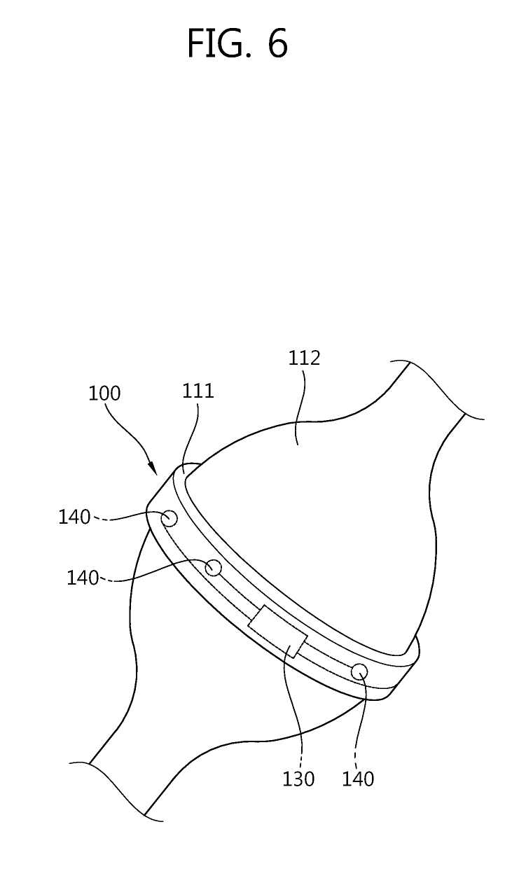

In this case, an endoscope is inserted though the same channel or different channels of the catheter together with the catheter to perform an operation while verifying an accurate implanting location. Accordingly, as illustrated in FIG. 6, the nerve root stimulator 100 is implanted in a state where each electrode 140 is in contact with the outside of the dorsal root ganglion. When the nerve root stimulator 100 is implanted in the dorsal root ganglion, an operation site is sutured, and the installation operation of the nerve root stimulator 100 ends.

In addition, in the implanting method of the nerve root stimulator 100, the catheter is inserted a spinal path through a sacral hiatus of a hip of the patient, and when the catheter reaches a location where the nerve root stimulator 100 is installed, the nerve root stimulator 100 is inserted through the channel of the catheter to be implanted at the installation location. In the method, since only a very fine part of the hip is cut, it is possible to further minimize a surgical scar and a tissue damage of the human body.

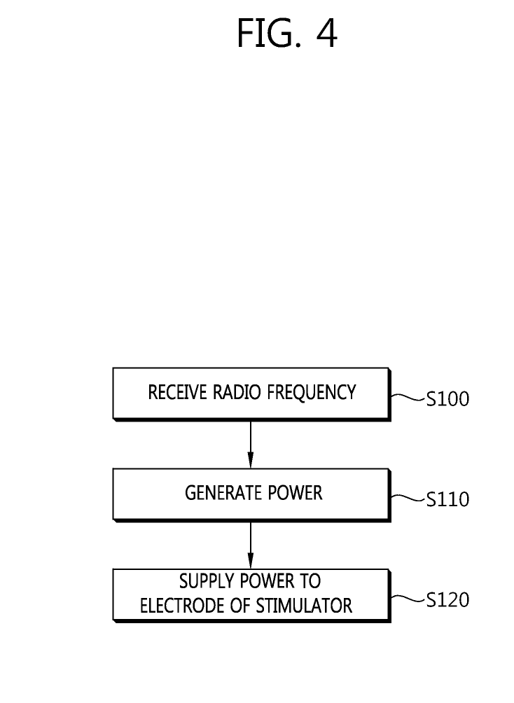

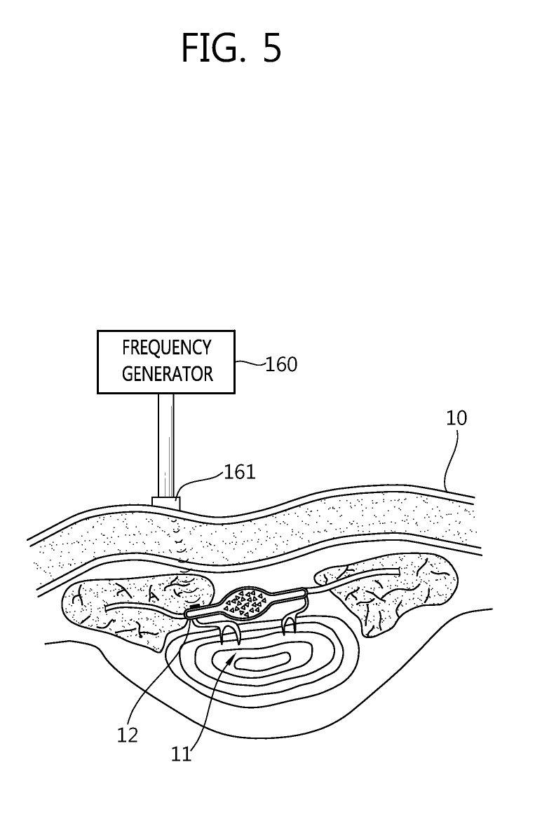

FIG. 4 is a flowchart illustrating a method for operating a nerve root stimulator according to another exemplary embodiment of the present invention, and FIG. 5 is a diagram for describing an operation state of a nerve root stimulator according to another exemplary embodiment of the present invention.

As illustrated in FIGS. 4 and 5, in a method of performing treatment by using the nerve root stimulator 100, a frequency oscillating tip 161 of a radio frequency oscillator 160 contacts the back portion of a human body 10. In addition, the radio frequency is oscillated to the inside of the human body 10. In this case, the oscillated radio frequency uses a frequency band which is harmless to the human body 10. In addition, transmission power may be selectively controlled according to power required in the nerve root stimulator 100.

As a result, the generation power of the nerve root stimulator 100 may be calculated by Equation 1.

$$Ps = {Pg ( \frac{λ}{4 π r} )}^{2} GsGg \qquad [ Equation 1 ]$$

Here, Ps is reception power of the nerve root stimulator 100, Pg is transmission power of a radio frequency oscillator, λ is a wavelength of a wave, r is a distance from the frequency oscillator to the nerve root stimulator 100, Gs is gain of the nerve root stimulator 100, and Gr is gain of the frequency oscillator 160.

That is, by verifying the distance between the radio frequency oscillator 160 and the nerve root stimulator 100 and the power consumption required in the nerve root stimulator 100, the radio frequency which may obtain the power required in the nerve root stimulator 100 may be transmitted to the nerve root stimulator 100.

In addition, the current required in the nerve root stimulator 100 may be 0.5 V to 40 V. Accordingly, the nerve root stimulator 100 finally receives the power of 10 to 40 dBm to obtain the power for operating the nerve root stimulator 100. In addition, in this case, the usable radio frequency may be 0.5 to 20 MHz, and further, the range of the frequency, the amplitude of the current, and the like may be variably controlled and used if necessary.

The nerve root stimulator 100 receives the radio frequency wirelessly transmitted by the antenna 150 (S100), and generates power by using the received radio frequency (S110). In addition, the power is applied to the electrode 140 of the nerve root stimulator 100 by using the power (S120) to stimulate a neural stimulation site of the human body 10 such as a nerve root 11 and a dorsal root ganglion 12. In this case, the electrode 140 may stimulate the neural stimulation site of the human body 10 such as the nerve root 11 and the dorsal root ganglion 12 by a bipolar method, but as another example, a plurality of electrodes 140 may stimulate the neural stimulation site by a monopolar method and a combination method of monopolar and bipolar.

Meanwhile, a plurality of nerve root stimulators 100 may be fixedly installed to a plurality of different dorsal root ganglions, respectively. Each of the nerve root stimulators 100 wirelessly receives the frequency to selectively stimulate each dorsal root ganglion.

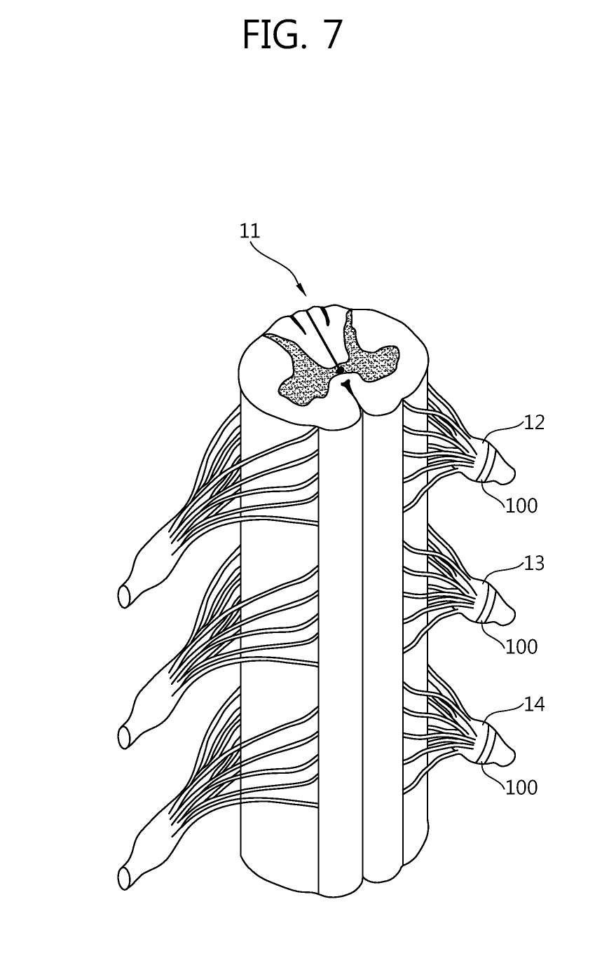

FIG. 7 is a diagram for describing an example of a using state of a nerve root stimulator according to another exemplary embodiment of the present invention. As illustrated in FIG. 7, dorsal root ganglions 11, 12, and 13 are connected to different tissues of the human body, respectively to control pain and movement signals of each tissue. Accordingly, the nerve root stimulator 100 is installed at each of the dorsal root ganglions 11, 12, and 13, and as a result, only the nerve root stimulator 100 may be controlled to operate at a desired location.

To this end, although not illustrated, the nerve root stimulator 100 may include a modulator. The modulator is mounted on the main chip 130 together to transmit a necessary signal to the outside in the main chip 130. Accordingly, when the radio frequency is transmitted to the nerve root stimulator 100, the demodulator 134 demodulates a command loaded in the received radio frequency to transfer the command to the control unit 133.

In this case, if a command that gives a command so that only a nerve root stimulator 100 performs the stimulation at a predetermined site is included in the transferred signal, only the corresponding nerve root stimulator 100 perform the stimulation and the rest of the nerve root stimulators 100 may not perform the stimulation. That is, in the case where the plurality of nerve root stimulators 100 are installed at the dorsal root ganglions 11, 12, and 13 in the human body, only the nerve root stimulator 100 installed at a predetermined dorsal root ganglion 11, 12, or 13 performs the stimulation and the stimulation is performed only at a predetermined pain site of the patient to perform treatment of pains and movement disorders.

Meanwhile, in a method of using the nerve root stimulator 100 according to the exemplary embodiment of the present invention, as a method of irradiating the radio frequency to the nerve root stimulator 100, the patient contacts the frequency oscillator to the back portion outside the human body to irradiate the radio frequency, but when the patient carries other different terminals, the treatment may be performed.

That is, a radio portable communication terminal which the patient carries is configured to oscillate the required radio frequency, and if the patient requests, the required radio frequency may be irradiated to the nerve root stimulator 100. That is, when the patient determines that the pain or the movement disorder exists by himself while carrying the radio portable communication terminal, the radio frequency for treatment is irradiated to the nerve root stimulator 100 by operating the radio portable communication terminal, and as a result, the self-treatment may be easily performed in any place at all times.

It should not be appreciated that the exemplary embodiments of the present invention described above and illustrated in the drawings limit the technical spirit of the present invention. The protection scope of the present invention is limited by only matters described in the claims and the technical spirit of the present invention can be modified and changed in various forms by those skilled in the art. Accordingly, the modification and the change will belong to the protection scope of the present invention as long as the modification and change are apparent to those skilled in the art.

What is claimed is:

1. A nerve root stimulator, comprising:
a body part installed in a spinal nerve root of a human body;
an antenna provided in the body part and receiving a radio frequency irradiated outside the human body;
a power generating unit generating power by using the radio frequency received to the antenna; and
an electrode electrically stimulating the spinal nerve root by using the power generated in the power generating unit.

2. The nerve root stimulator of claim 1, wherein the power generating unit comprises a rectifying unit rectifying the radio frequency received to the antenna to a direct current and a switching unit controlling the power provided to the electrode.

3. The nerve root stimulator of claim 1, wherein a plurality of electrodes is provided, and the power generating unit provides power having different sizes to the plurality of electrodes.

4. The nerve root stimulator claim 1, further comprising:
a demodulator demodulating a transmission signal at the radio frequency received to the antenna and a control unit controlling an operation of the power generating unit by using data transferred from the demodulator.

5. The nerve root stimulator of claim 1, wherein a plurality of antennas extended in a length direction of the body part is included.

6. A method for operating a nerve root stimulator, comprising:
receiving a radio frequency by a nerve root stimulator installed in a spinal nerve root of a human body;
generating power by using the received radio frequency; and
applying a current to an electrode of the nerve root stimulator contacting the nerve root by using the power.

7. The method of claim 6, wherein a plurality of electrodes is provided, and currents having different intensities are applied to the plurality of electrodes.

8. The method of claim 6, wherein a transmission signal is demodulated at the received radio frequency to control the operation of the nerve root stimulator.

9. The method of claim 6, wherein the nerve root stimulator is installed at a dorsal root ganglion.

10. A method for operating a nerve root stimulator, comprising:
receiving a radio frequency by a plurality of nerve root stimulators installed at a plurality of locations in a spinal nerve root in a human body;
demodulating an identification signal from the radio frequency by the plurality of nerve root stimulators;
verifying an unique identifier from the identification signal by the plurality of nerve root stimulators;
generating power by using the received radio frequency by the nerve root stimulator identified by the unique identifier among the plurality of nerve root stimulators; and
applying a current to an electrode of the identified nerve root stimulators by using the power.

11. The method of claim 10, wherein a plurality of electrodes is provided, and currents having different intensities are applied to the plurality of electrodes.

12. The method of claim 10, wherein a transmission signal is demodulated at the received radio frequency to control the operation of the nerve root stimulator.

13. The method of claim 10, wherein the nerve root stimulator is installed at a dorsal root ganglion.
